(19) United States
(12) Patent Application Publication
(10) Pub. No.: US 2022/0115086 A1
(43) Pub. Date: Apr. 14, 2022

(54) ALTERING PROTEIN-LIGAND STRUCTURE ACCORDING TO PROTEIN-LIGAND BINDING AFFINITY

(71) Applicant: International Business Machines Corporation, Armonk, NY (US)

(72) Inventors: Giacomo Domeniconi, White Plains, NY (US); Leili Zhang, White Plains, NY (US); Guojing Cong, Ossining, NY (US); Chih-Chieh Yang, White Plains, NY (US); Ruhong Zhou, Fort Lee, NJ (US)

(21) Appl. No.: 17/065,548

(22) Filed: Oct. 8, 2020

(51) Int. Cl. G16B 15/20; G16C 20/20

(52) U.S. Cl. CPC G16B 15/20; G16C 20/20

(57) ABSTRACT

Altering protein-ligand structures by generating molecular trajectory data for a protein-ligand structure, determining a molecular level binding affinity according to the molecular trajectory data, determining an atom level binding affinity for a first atom of the protein-ligand structure according to the molecular trajectory data, determining a correlation between the atom level and the molecular level binding affinities, and altering the protein-ligand structure according to the correlation.

### BACKGROUND

The disclosure relates generally to modifying protein-ligand structures according to protein-ligand binding affinities. The disclosure relates particularly to modifying protein-ligand structures according to anomalous protein-ligand binding affinities.

Drug discovery methodologies include experimentation where molecular constituents are randomly replaced with alternatives without clear predictions as to the impact of the replacement. Using molecular dynamics to evaluate structures provides a lower cost alternative for evaluating structures.

### SUMMARY

The following presents a summary to provide a basic understanding of one or more embodiments of the disclosure. This summary is not intended to identify key or critical elements or delineate any scope of the particular embodiments or any scope of the claims. Its sole purpose is to present concepts in a simplified form as a prelude to the more detailed description that is presented later. In one or more embodiments described herein, devices, systems, computer-implemented methods, apparatuses and/or computer program products enable the evaluation and alteration of protein-ligand structures.

Aspects of the invention disclose methods, systems and computer readable media associated with altering protein-ligand structures by generating molecular trajectory data for a protein-ligand structure, determining a molecular level binding affinity according to the molecular trajectory data, determining an atom level binding affinity for a first atom of the protein-ligand structure according to the molecular trajectory data, determining a correlation between the atom level and the molecular level binding affinities, and altering the protein-ligand structure according to the correlation.

### BRIEF DESCRIPTION OF THE DRAWINGS

Through the more detailed description of some embodiments of the present disclosure in the accompanying drawings, the above and other objects, features and advantages of the present disclosure will become more apparent, wherein the same reference generally refers to the same components in the embodiments of the present disclosure.

FIG. 1 provides a schematic illustration of a computing environment, according to an embodiment of the invention.

FIG. 2 provides a flowchart depicting an operational sequence, according to an embodiment of the invention.

FIG. 3 depicts the chemical structure of a compound modified, according to an embodiment of the invention.

FIG. 4 depicts the chemical structure of a compound modified, according to an embodiment of the invention.

FIG. 5 depicts a cloud computing environment, according to an embodiment of the invention.

FIG. 6 depicts abstraction model layers, according to an embodiment of the invention.

### DETAILED DESCRIPTION

Some embodiments will be described in more detail with reference to the accompanying drawings, in which the embodiments of the present disclosure have been illustrated. However, the present disclosure can be implemented in various manners, and thus should not be construed to be limited to the embodiments disclosed herein.

In an embodiment, one or more components of the system can employ hardware and/or software to solve problems that are highly technical in nature (e.g., generating molecular dynamics trajectory data for an protein-ligand structures, determining molecular level binding affinities, determining atom level binding affinities, correlating molecular level and atom level binding affinities, altering the protein-ligand structure according to the correlation, etc.). These solutions are not abstract and cannot be performed as a set of mental acts by a human due to the processing capabilities needed to facilitate altering protein-ligand structures, for example. Further, some of the processes performed may be performed by a specialized computer for carrying out defined tasks related to memory operations. For example, a specialized computer can be employed to carry out tasks related to suggest protein-ligand structure alternatives or the like.

As used herein, protein-ligand structure refers to a computer simulated structure representing a protein-ligand complex. References to altering a protein-ligand structure relate to identifying an alternative protein-ligand structure having superior binding properties between the protein and ligand.

Pharmaceutical discovery represents an 80-billion-dollar business. Many discovery pathways rely upon experimentation with different compound structures, or evaluation of randomized protein-ligand structures using molecular modeling software. Disclosed embodiments provide a structured methodology for identifying improvements to initial protein-ligand structures. Disclosed embodiments identify those portions of an existing structure which represent the greatest opportunity for improving efficacy as well as alternatives for the replacement. Disclosed embodiments may be applied in the identification of lead candidates in pharmaceutical research efforts. Disclosed embodiments provide for the evaluation of the relative binding affinities of an entire ligand-protein combination as well as the binding affinities of individual atoms of the ligand. Increasing the overall binding affinity of the ligand-protein combination by selectively replacing individual ligand atoms according to the relative binding affinity of the atom and overall ligand improves the efficacy of the protein-ligand structure by improving the ligand-protein interaction strength.

In an embodiment, the method considers an initial protein-ligand structure and variants of the protein-ligand structure. In this embodiment, the method identifies a binding target within the structure between a protein and a ligand of the structure. The method considers structures having differing constituents as the ligand binding portion of the structure. The method prepares molecular dynamic (MD) simulations of the considered structures for each of the initial structure and those structures having alternative ligand components. The method may conduct a single molecular dynamics simulation for each structure, or the method may conduct a plurality of simulations for each structure and calculate averages of the molecular trajectory data from the set of simulations for each structure.

The MD simulations provide data relating the trajectories and relative locations of protein-ligand structure constituents due to intramolecular forces acting upon the overall protein-ligand structure over time. The MD simulations capture the relationships between constituents due to constituent kinetics arising from interaction energies and binding energies of the constituents. Multiple MD simulations of a single structure yield different constituent trajectory results due to the probabilistic nature of the dynamics between constituents. In an embodiment, the method utilizes nanoscale molecular dynamics (NAMD) software to simulate molecule constituents' trajectories. The simulations yield data sets including temporal sequences capturing the relative positions of the constituents of the protein-ligand structure over time.

In an embodiment, the method analyzes the trajectory data in terms of the dynamics of the entire ligand of the molecule. In this embodiment, the method uses a clustering methodology, such as quality threshold (QT) clustering to group the ligand dynamics data. The method considers the dynamics of the set of atoms of the ligand as a group. The method calculates a root mean square displacement (RMSD) for the set of ligand atoms relative to the set of protein atoms of the overall molecule. The QT clustering then groups the RMSD data according to nearest neighbor distance, bounding each cluster according to a defined threshold distance and the minimum number of data points for a cluster.

The method determines candidate clusters taking each data point of the input data set as the starting point for a cluster. The method evaluates the set of candidate clusters and selects the candidate cluster having the largest number of members and satisfying the distance threshold and minimum member requirements. The data points of this cluster are removed from consideration and the method repeats the clustering process with the remaining data points, creating candidate clusters and selecting the best candidate, then removing the data points of that candidate from further consideration. Clustering continues until all input data points are assigned to a cluster. Clustering the trajectory data provides an indication of the relative freedom of movement between the ligand and the protein. The relative freedom of movement provides an indication of the binding affinity between the ligand as a whole and the protein. Clusters having relatively high freedom of movement indicated by larger trajectory distances, tend to have lower binding affinities and clusters having relatively low freedom of movement indicated by small trajectory distances tend to have higher binding affinities

The input data set includes trajectories for each atom in the overall molecule. The data may be analyzed in terms of the relative positions of the atoms of the ligand with the positions of the atoms of the protein. The data may be considered as an N×M matrix, where N represents the atoms of the ligand and M represents the atoms of the protein. Analysis of the data considers the contacts between pairs of atoms which are less than the threshold distance apart. For analyzing the ligand as a whole, the method considers the set of protein atoms to be aligned in location and evaluates the trajectory of the set of ligand atoms with respect to the aligned location of the protein set. In an embodiment, the method determines atom level binding affinity for the individual atoms of the ligand with respect to the protein.

In an embodiment, the complexity of the ligand atom-protein analysis using the N×M matrix may be reduced by subtyping the ligand atoms according to common physical properties. In this embodiment, the method categorizes ligand atoms according to QT clustering of van der Waals parameters such as well depth (c) and atom size for the different ligand atoms. As an example, the method divides the ligand atoms (e.g., H, C, O, N, S, P, F, Cl, etc.) into 46 subtypes based upon Chemistry at Harvard Molecular Mechanics (CHARMM) force field data using 10% of ε and rₘᵢₙ in the Lennard-Jones potential form as the QT threshold. The Lennard-Jones potential approximates the intermolecular electrical potential between a pair of molecules. Exemplary atom subtypes include carbonyl carbons, aromatic carbons, aliphatic carbons, hydroxyl oxygens, carbonyl oxygens, and disulfide sulfurs. (Note: the term “CHARMM” may be subject to trademark rights in various jurisdictions throughout the world and are used here only in reference to the products or services properly denominated by the marks to the extent that such trademark rights may exist.)

After subtyping the ligand atoms, the method alters the analysis problem from consideration of the N×M matrix to consideration of an S (number of subtypes)×M matrix. For each subtype, the method considers the vector of distances for that subtype to the M atoms of the protein. The method clusters the MD trajectory data in terms of the S×M matrix for the atoms of the ligand. The method groups MD data frames having similar bound and unbound states as indicated by the relative ligand atom-protein positions. In this embodiment, the method uses the clustering algorithm HDBSCAN (hierarchical density-based spatial clustering of applications with noise) to group the MD data frames. The method clusters the embedded M sized vectors for each subtype from the MD trajectory. The clustering algorithm may be applied on the M sized vectors or on a lower dimensional representation created with algorithms such as autoencoder neural network, variational autoencoder neural network, generative adversarial network, and principle component analysis. The clustering output provides an indication of the relative freedom of movement of the individual atoms or atom subtypes, with respect to the protein. The relative freedom of movement of the atoms or atom subtypes provides an indication of the binding affinity of the atoms or subtypes to the protein. Atoms and subtypes having relatively higher freedom of movement indicated by larger distances in the trajectory data, tend to have lower binding affinities.

The method may conduct the clustering analysis of the molecular ligand level and the atom or subtype levels using input MD trajectory data from a single simulation of the particular protein-ligand combination, or the method may use an input data set including the trajectory results from a plurality of simulations of the same protein-ligand combination.

In an embodiment, the method correlates the molecular level binding affinity of the entire ligand with the protein and the binding affinities of the individual ligand atoms with the protein. The method considers the relative binding affinities of individual ligand atoms and the ligand as a whole, rather than absolute affinities. In this embodiment, the method compares the clustering data for the ligand as a whole with the clustering data for the individual ligand atoms or atom subtypes. As an example, the method uses cosine similarity (CosSim) between the respective sets of clustered trajectory data. Differences in the CosSim values for different atoms and subtypes indicate differences in relative binding affinity for the respective atoms and subtypes. As an example, a structure has an average CosSim for all ligand atom subtypes X, Y, and Z of 0.903. Subtype X has a CosSim of 0.734, and subtype Y has a CosSim of 0.963. For the example, subtype X has a lower than average CosSim, indicating a lower than average binding affinity to the protein and therefore weakens the overall ligand-protein binding. Subtype Y has a higher than average CosSim indicating a higher than average binding affinity to the protein, and therefore strengthens the overall ligand-protein binding.

In an embodiment, the method considers multiple alternative protein-ligand structures. Each alternative structure includes one or more alternative ligand constituents. For example, for an initial ligand comprising subtype X, the method also considers and generates MD trajectory simulation and clustered data for ligands having subtype X′, X″, etc. Considering the alternative ligands with different subtypes enables the method to identify alternatives to subtypes having lower than average binding affinities with the accompanying deleterious impact upon the efficacy of the protein-ligand structure. For example, whereas subtype X (corresponding to CO) has a CosSim of 0.734, subtype X′ (corresponding to CHCl) may be found to have a higher CosSim than 0.734, and mutating the ligand of the protein-ligand structure by replacing the subtype X with subtype X′, strengthens the ligand-protein bond and improves the associated overall molecular efficacy.

In an embodiment, the method validates or verifies the benefits of a mutation identified using the alternative structure cluster data. The method conducts free energy perturbation (FEP) simulations of the original and mutated structures to validate that the mutation reduces the free energy of the overall structure, indicating a strengthened ligand-protein interaction.

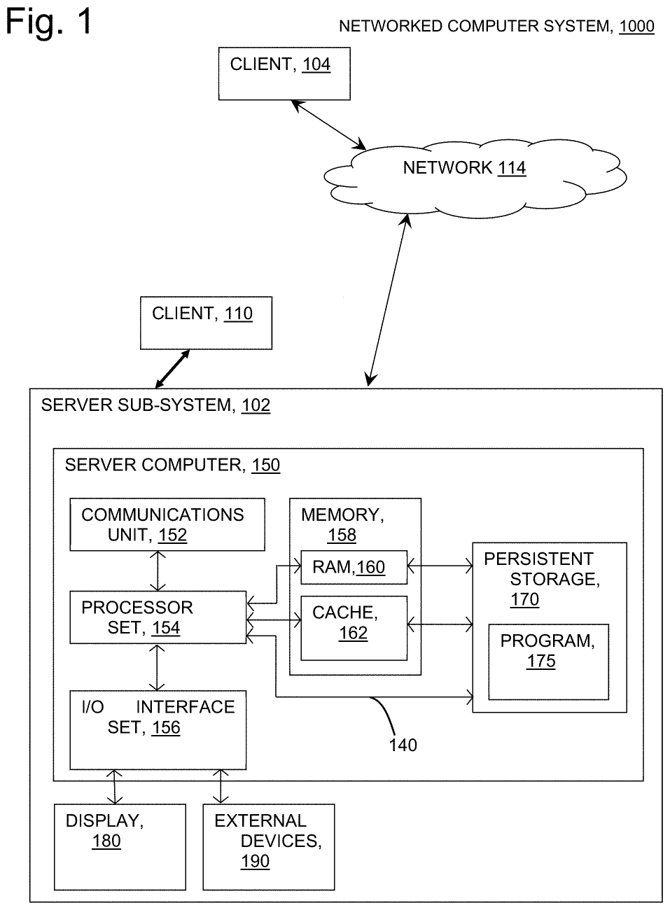

FIG. 1 provides a schematic illustration of exemplary network resources associated with practicing the disclosed inventions. The inventions may be practiced in the processors of any of the disclosed elements which process an instruction stream. As shown in the figure, a networked client device 110 connects wirelessly to server sub-system 102. Client device 104 connects wirelessly to server sub-system 102 via network 114. Client devices 104 and 110 comprise protein-ligand structure analysis programs (not shown) together with sufficient computing resources (processor, memory, network communications hardware) to execute the program. Connecting client devices 104 and/or 110 to the server sub-system 102 enables the operational steps of the method to be carried out using the extended computing resource environment provided by the server sub-system 102, including the use of edge cloud and/or cloud resources for the computationally intensive aspects of the MD simulations, clustering and correlating portions of the method. As shown in FIG. 1, server sub-system 102 comprises a server computer 150. FIG. 1 depicts a block diagram of components of server computer 150 within a networked computer system 1000, in accordance with an embodiment of the present invention. It should be appreciated that FIG. 1 provides only an illustration of one implementation and does not imply any limitations with regard to the environments in which different embodiments can be implemented. Many modifications to the depicted environment can be made.

Server computer 150 can include processor(s) 154, memory 158, persistent storage 170, communications unit 152, input/output (I/O) interface(s) 156, and communications fabric 140. Communications fabric 140 provides communications between cache 162, memory 158, persistent storage 170, communications unit 152, and input/output (I/O) interface(s) 156. Communications fabric 140 can be implemented with any architecture designed for passing data and/or control information between processors (such as microprocessors, communications and network processors, etc.), system memory, peripheral devices, and any other hardware components within a system. For example, communications fabric 140 can be implemented with one or more buses.

Memory 158 and persistent storage 170 are computer readable storage media. In this embodiment, memory 158 includes random access memory (RAM) 160. In general, memory 158 can include any suitable volatile or non-volatile computer readable storage media. Cache 162 is a fast memory that enhances the performance of processor(s) 154 by holding recently accessed data, and data near recently accessed data, from memory 158.

Program instructions and data used to practice embodiments of the present invention, e.g., the protein-ligand structure analysis program 175, are stored in persistent storage 170 for execution and/or access by one or more of the respective processor(s) 154 of server computer 150 via cache 162. In this embodiment, persistent storage 170 includes a magnetic hard disk drive. Alternatively, or in addition to a magnetic hard disk drive, persistent storage 170 can include a solid-state hard drive, a semiconductor storage device, a read-only memory (ROM), an erasable programmable read-only memory (EPROM), a flash memory, or any other computer readable storage media that is capable of storing program instructions or digital information.

The media used by persistent storage 170 may also be removable. For example, a removable hard drive may be used for persistent storage 170. Other examples include optical and magnetic disks, thumb drives, and smart cards that are inserted into a drive for transfer onto another computer readable storage medium that is also part of persistent storage 170.

Communications unit 152, in these examples, provides for communications with other data processing systems or devices, including resources of client computing devices 104 and 110. In these examples, communications unit 152 includes one or more network interface cards. Communications unit 152 may provide communications through the use of either or both physical and wireless communications links. Software distribution programs, and other programs and data used for implementation of the present invention, may be downloaded to persistent storage 170 of server computer 150 through communications unit 152.

I/O interface(s) 156 allows for input and output of data with other devices that may be connected to server computer 150. For example, I/O interface(s) 156 may provide a connection to external device(s) 190 such as a keyboard, a keypad, a touch screen, a microphone, a digital camera, and/or some other suitable input device. External device(s) 190 can also include portable computer readable storage media such as, for example, thumb drives, portable optical or magnetic disks, and memory cards. Software and data used to practice embodiments of the present invention, e.g., protein-ligand structure analysis program 175 on server computer 150, can be stored on such portable computer readable storage media and can be loaded onto persistent storage 170 via I/O interface(s) 156. I/O interface(s) 156 also connect to a display 180.

Display 180 provides a mechanism to display data to a user and may be, for example, a computer monitor. Display 180 can also function as a touch screen, such as a display of a tablet computer.

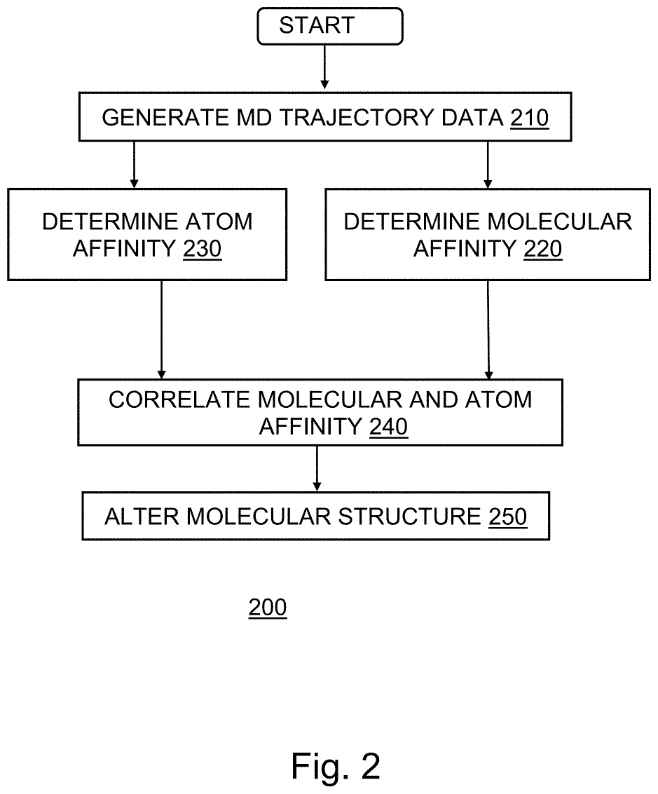

FIG. 2 provides a flowchart 200, illustrating exemplary activities associated with the practice of the disclosure. After program start, at block 210, the method of protein-ligand structure analysis program 175 generates MD trajectory data for an initial protein-ligand structure as well as for mutated versions of the initial protein-ligand structure. In an embodiment, the mutated structures comprise known alternative ligand constituents replacing ligand constituents of the original protein-ligand structure.

At block 220, the method of protein-ligand structure analysis program 175 determines molecular level binding affinities according to the molecular trajectory data of the MD simulations. The method considers the set of atoms comprising the ligand of the protein-ligand structure as a whole in relationship to the protein of the structure. The method considers the location of the protein as fixed or flexible and evaluates the location of the set of ligand atoms with respect to the aligned location of the protein atoms. The method utilizes a clustering algorithm such as QT clustering to group the ligand level trajectory data. The method relates the data clusters to the binding affinity of the ligand to the protein as having an inverse relationship to the freedom of movement of the ligand with regard to the protein as determined from the trajectory data analysis.

At block 230, the method determines atom level binding affinities for the individual atoms of the ligand with respect to the protein of the protein-ligand structure. The method may evaluate each ligand atom relative to the protein or the method may reduce the set of ligand atoms by categorizing the ligand atoms as atom subtypes according to the physical properties of the respective atoms such as the partial charge, atom size, and force fields, such as the force fields provided by CHARMM. The method clusters the ligand atoms according to physical properties using a clustering algorithm such as QT clustering and yields a set of atom subtypes corresponding to the set of ligand atoms. This clustering transforms the set of N ligand atoms to a set of S atom subtypes and enables analysis of the ligand atoms in terms of grouped subtypes rather than as individual atoms. The method then evaluates the trajectory data of the MD simulation using the S×M matrix (where M represents the number of atoms in the protein of the structure). This analysis yields a set of distance vectors of size M, one vector for each of the S atom subtypes. The method creates low dimensionality vector data using an algorithm such as an autoencoder neural network, a variational autoencoder neural network, principle components analysis, a generative adversarial neural network, etc. Then the method clusters the atoms with a clustering algorithm, such as DBSCAN, HDBSCAN, agglomerative clustering, etc. The method associates atom level binding affinities for the ligand atoms with the clustered MD trajectory data as the inverse of the atom freedom of movement with respect to the protein indicated by the clustered trajectory data.

At block 240, the method correlates the ligand level and atom level binding affinities using a similarity measure such as Cosine Similarity (CosSim) between the respective sets of clustered MD trajectory data. The method determines the similarity between each atom subtype and the overall ligand as well as an average similarity across the set of subtypes with respect to the overall ligand. Subtype CosSim values less than the subtype average CosSim indicate atom subtypes having a weaker affinity than the overall ligand while subtype CosSim values above the average indicate subtypes having a stringer affinity for the protein than the overall ligand.

CosSim values below the average for the individual atom subtypes represent an opportunity to improve the overall ligand-protein binding affinity and thereby improve the efficacy of the protein-ligand structure.

At block 250 the method alters the original protein-ligand structure. Using atom or subtype correlations associated with mutated versions of the original protein-ligand structure, the method identifies ligand constituent atoms or subtypes having CosSim values greater than the lowest ligand atoms or subtypes. The method generates a mutated structure including alternative subtypes or atoms replacing the original low CosSim values ligand constituents.

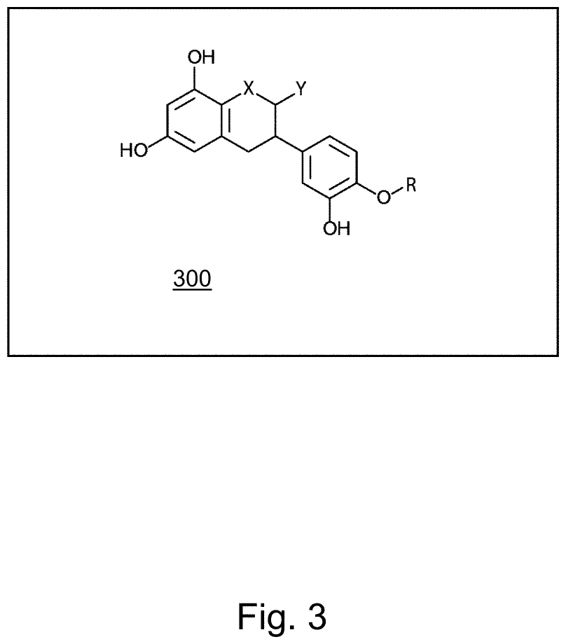

FIG. 3 illustrates the chemical structure 300 of an isovanillyl sweetener. As shown in the figure, the ligand which bonds to the protein of the overall structure, includes subtype locations X and Y. For this example, the method simulates the ligand molecule using CO (subtype 8, aromatic carbonyls) as location X and CO (subtype 10, aliphatic carbonyls) as location Y. The method determines that subtype 8 has a lower CosSim value (0.869) than the average for the ligand (0.900) while subtype 10 has a higher value (0.903) than the average for the ligand.

FEP simulations were conducted for the original structure, where location X includes subtype 8, and an altered structure, where subtype 8 and was replaced with CH₂, subtype 12, indicates a drop in free energy for the protein-ligand structure of −3.8±1.0 kcal/mol verifying the increase in binding affinity predicted by the clustering data analysis. Altering the CO to CH₂ for subtype location X, yields an improved efficacy in the form of an approximate 600-fold increase in sweetness.

Conducting an FEP simulation of the original structure where Y includes subtype 10, and the altered structures, replacing subtype 10 at Y with CH₂ subtype 12 at Y, indicates no significant change in free energy for the protein-ligand structure (−1.1±1.4 kcal/mol), and little change in the sweetness of the altered compound.

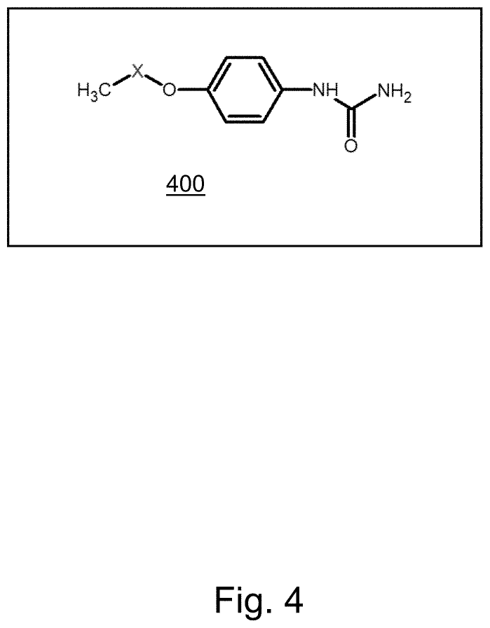

FIG. 4 illustrates the chemical structure 400 of another artificial sweetener, where X represents the location of the subtype being considered. In the original ligand compound (Dulcin), X is a methylene group (CH₂). The method evaluated the structure with each of CH₂ (original) and CHCl (mutated) at the X location using MD simulations. For the original structure, the method determined that the CH₂ CosSim value of 0.873 was less than the ligand's average subtype value of 0.947. Subtypes having a CosSim value less than the average have a weaker bond than the overall ligand. This weaker than average bond indicates that replacement of the CH₂ presents an opportunity for improving the binding affinity and overall efficacy of the protein-ligand structure. From the alternative structure MD data, the method determined that altering the CH₂ to CHCl will increase the overall binding affinity and efficacy of the protein-ligand structure. The method conducted FEP simulations of the original and altered structures and determined that the altered structure had a free energy reduction of −2.4±1.0 kcal/mol, validating the prediction that CHCl would improve the overall binding affinity. Analysis of the altered protein-ligand structure indicated an approximate 57-fold increase in sweetness.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

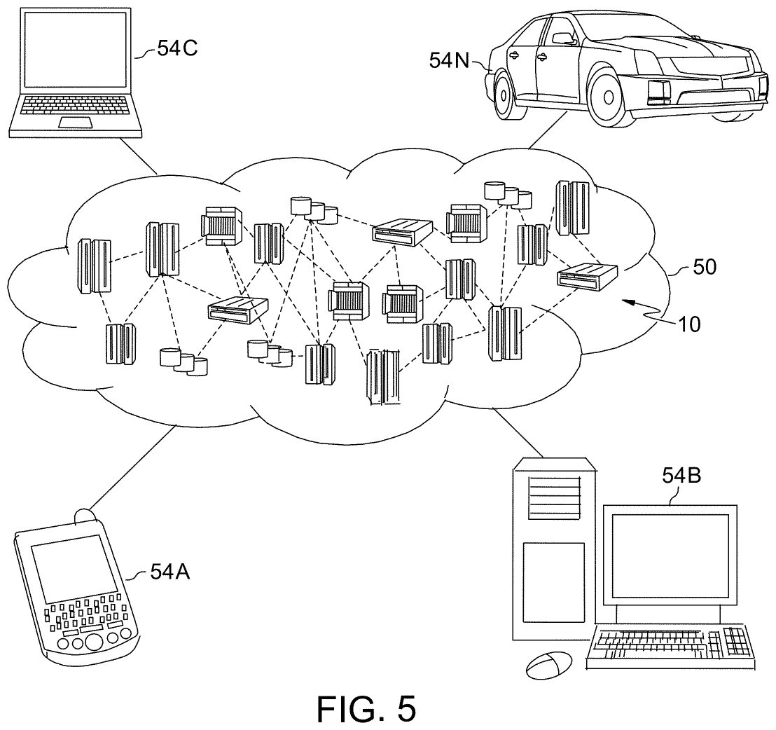

Referring now to FIG. 5, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 5 are intended to be illustrative only and that computing nodes 10 and cloud computing environment 50 can communicate with any type of computerized device over any type of network and/or network addressable connection (e.g., using a web browser).

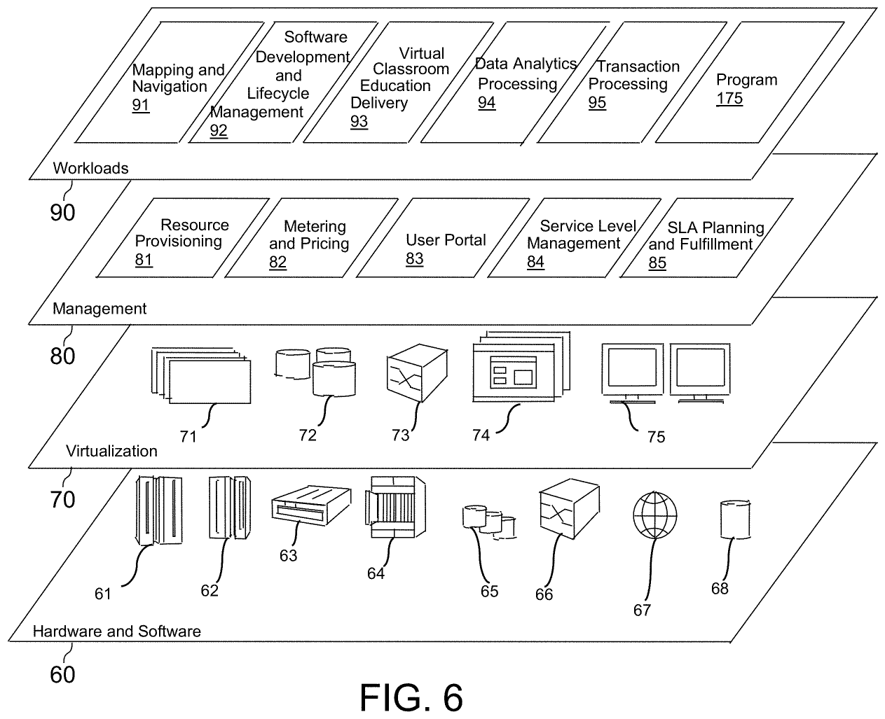

Referring now to FIG. 6, a set of functional abstraction layers provided by cloud computing environment 50 (FIG. 5) is shown. It should be understood in advance that the components, layers, and functions shown in FIG. 6 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture-based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and protein-ligand structure analysis program 175.

The present invention may be a system, a method, and/or a computer program product at any possible technical detail level of integration. The invention may be beneficially practiced in any system, single or parallel, which processes an instruction stream. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, or computer readable storage device, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions collectively stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer implemented method for altering protein-ligand structures, the method comprising:
generating molecular trajectory data for a protein-ligand structure;
determining a molecular level binding affinity according to the molecular trajectory data;
determining an atom level binding affinity for a first atom of the protein-ligand structure according to the molecular trajectory data;
determining a correlation between the atom level and the molecular level binding affinities; and
altering the protein-ligand structure according to the correlation.

2. The computer implemented method according to claim 1, further comprising:
determining atom level binding affinities for a second atom; and
altering the protein-ligand structure by replacing the first atom with the second atom.

3. The computer implemented method according to claim 1, wherein determining an atom level binding affinity comprises determining an atom level binding affinity of an atom subtype.

4. The computer implemented method according to claim 1, wherein:
determining the molecular level binding affinity according to the molecular trajectory data comprises determining molecular trajectory data clusters;
determining the atom level binding affinity for a first atom of the protein-ligand structure according to the molecular trajectory data comprises determining atomic trajectory data clusters; and
determining the correlation between the atom level binding affinity and the molecular level binding affinity comprises determining a correlation between the molecular trajectory data clusters and the atomic trajectory data clusters.

5. The computer implemented method according to claim 1, further comprising verifying the correlation using a free energy perturbation simulation of the protein-ligand structure.

6. The computer implemented method according to claim 1, wherein determining the molecular level binding affinity according to the molecular trajectory data comprises determining a root mean square displacement of a ligand of the protein-ligand structure from a protein of the protein-ligand structure according to the molecular trajectory data.

7. The computer implemented method according to claim 1, wherein determining the atom level binding affinity for a first atom of the protein-ligand structure according to the molecular trajectory data comprises:
categorizing atoms of a ligand of the protein-ligand structure as atom subtypes;
generating vectors comprising the atom subtypes and atoms of a protein of the protein-ligand structure;
determining distances according to the vectors;
determining data clusters according to the distances; and
determining atom level binding affinities according to the clusters.

8. A computer program product to alter protein-ligand structures, the computer program product comprising one or more computer readable storage devices and collectively stored program instructions on the one or more computer readable storage devices, the stored program instructions comprising:
program instructions to generate molecular trajectory data for a protein-ligand structure;
program instructions to determine a molecular level binding affinity according to the molecular trajectory data;
program instructions to determine an atom level binding affinity for a first atom of the protein-ligand structure according to the molecular trajectory data;
program instructions to determine a correlation between the atom level and the molecular level binding affinities; and
program instructions to alter the protein-ligand structure according to the correlation.

9. The computer program product according to claim 8, the stored program instructions further comprising:
program instructions to determine atom level binding affinities for a second atom; and
program instructions to alter the protein-ligand structure by replacing the first atom with the second atom.

10. The computer program product according to claim 8, wherein the program instructions to determine the atom level binding affinity comprise program instructions to determine an atom level binding affinity of an atom subtype.

11. The computer program product according to claim 8, wherein:
program instructions to determine the molecular level binding affinity according to the molecular trajectory data comprise program instructions to determine molecular trajectory data clusters;
program instructions to determine the atom level binding affinity for a first atom of the protein-ligand structure according to the molecular trajectory data comprise program instructions to determine atomic trajectory data clusters; and
program instructions to determine the correlation between the atom level binding affinity and the molecular level binding affinity comprise program instructions to determine a correlation between molecular trajectory data clusters and atomic level trajectory clusters.

12. The computer program product according to claim 8, the stored program instructions further comprising program instructions to verify the correlation using a free energy perturbation simulation of the protein-ligand structure.

13. The computer program product according to claim 8, wherein the stored program instructions determine the molecular level binding affinity according to the molecular trajectory data comprise program instructions to determine a root mean square displacement of a ligand of the protein-ligand structure from a protein of the protein-ligand structure according to the molecular trajectory data.

14. The computer program product according to claim 8, wherein program instructions to determine the atom level binding affinity for a first atom of the protein-ligand structure according to the molecular trajectory data comprise:
program instructions to categorize atoms of a ligand of the protein-ligand structure as atom subtypes;
program instructions to generate vectors comprising the atom subtypes and atoms of a protein of the protein-ligand structure;
program instructions to determine distances according to the vectors;
program instructions to determine data clusters according to the distances; and
program instructions to determine atom level binding affinities according to the clusters.

15. A computer system for altering protein-ligand structures, the computer system comprising:
one or more computer processors;
one or more computer readable storage devices; and
stored program instructions on the one or more computer readable storage devices for execution by the one or more computer processors, the stored program instructions comprising:
program instructions to generate molecular trajectory data for a protein-ligand structure;
program instructions to determine a molecular level binding affinity according to the molecular trajectory data;
program instructions to determine an atom level binding affinity for a first atom of the protein-ligand structure according to the molecular trajectory data;
program instructions to determine a correlation between the atom level and the molecular level binding affinities; and
program instructions to alter the protein-ligand structure according to the correlation.

16. The computer system according to claim 15, the stored program instructions further comprising:
program instructions to determine atom level binding affinities for a second atom; and
program instructions to alter the protein-ligand structure by replacing the first atom with the second atom.

17. The computer system according to claim 15, wherein:
program instructions to determine the molecular level binding affinity according to the molecular trajectory data comprise program instructions to determine molecular trajectory data clusters;
program instructions to determine the atom level binding affinity for a first atom of the protein-ligand structure according to the molecular trajectory data comprise program instructions to determine atomic trajectory data clusters; and
program instructions to determine the correlation between the atom level binding affinity and the molecular level binding affinity comprise program instructions to determine a correlation between molecular trajectory data clusters and atomic level trajectory clusters.

18. The computer system according to claim 15, the stored program instructions further comprising program instructions to verify the correlation using a free energy perturbation simulation of the protein-ligand structure.

19. The computer system according to claim 15, wherein the stored program instructions determine the molecular level binding affinity according to the molecular trajectory data comprise program instructions to determine a root mean square displacement of a ligand of the protein-ligand structure from a protein of the protein-ligand structure according to the molecular trajectory data.

20. The computer system according to claim 15, wherein program instructions to determine the atom level binding affinity for a first atom of the protein-ligand structure according to the molecular trajectory data comprise:
program instructions to categorize atoms of a ligand of the protein-ligand structure as atom subtypes;
program instructions to generate vectors comprising the atom subtypes and atoms of a protein of the protein-ligand structure;
program instructions to determine distances according to the vectors;
program instructions to determine data clusters according to the distances; and
program instructions to determine atom level binding affinities according to the clusters.
